(19) United States
(12) Patent Application Publication
(10) Pub. No.: US 2018/0282622 A1
(43) Pub. Date: Oct. 4, 2018

(54) CORE@SHELL PARTICLES COMPOSED OF SENSITIZING PERSISTENT PHOSPHOR CORE AND UPCONVERSION SHELL AND METHODS OF MAKING SAME

(71) Applicant: The Boardm of Regents of The University of Texas System, Austin, TX (US)

(72) Inventor: Yuanbing Mao, Edinburg, TX (US)

(21) Appl. No.: 15/524,416

(22) PCT Filed: Nov. 3, 2015
(86) PCT No.: PCT/US15/58784

§ 371 Date: May 4, 2017

Related U.S. Application Data
(60) Provisional application No. 62/074,833, filed on Nov. 4, 2014.

(51) Int. Cl. C09K 11/77; A61L 2/08; A61L 9/20; A61K 49/00; H01L 31/055; C02F 1/32; C02F 1/72

(52) U.S. Cl. CPC C09K 11/7708; A61L 2/088; A61L 9/205; A61K 49/0019; C02F 2305/08; C02F 1/32; C02F 1/725; C02F 2305/10; H01L 31/055

(57) ABSTRACT

Described herein are heterogeneous core@shell particles composed of a sensitizing persistent phosphor core and a lanthanide upconversion (UC) shell. The core@shell particles can be used for upconversion of visible light to ultraviolet light.

### BACKGROUND OF THE INVENTION

### 1. Field of the Invention

The invention generally relates to upconverting particles. More particularly, the invention related to upconverting particles capable of converting visible light to ultraviolet light.

### 2. Description of the Relevant Art

Different from fluorescence and phosphorescence, persistent luminescence is a phenomenon involving energy traps (such as electron or hole trap) in a material which are filled during excitation. After the end of excitation, the stored energy is gradually released to emitter centers which emit light usually by a fluorescence-like mechanism. The result is the material will glow in the dark after excited with UV or visible light. For example, trivalent chromium, Cr³⁺, is a favorable long-persistent luminescent center in solids under visible light excitation because of its broadband emission (650-1600 nm) due to the spin-allowed ⁴T₂/²E→⁴A₂ transition, which strongly depends on the crystal-field environment of the host lattices. Zinc gallogermanates are excellent hosts for achieving the long-persistent luminescence of Cr³⁺ because of the excellent ability of Cr³⁺ ions to substitute Ga³⁺ ions in distorted octahedral sites and the suitable host crystal-field strength. So far, long-persistent phosphors are mainly used in safety signs, watch dials, decorative objects and toys. They have also been used as probes in small animal optical imaging. However, long-persistent phosphors have not been used as sensitizers, not to mention for enhancing upconversion (“UC”) luminescence of lanthanide upconverters.

UC luminescence is an anti-Stokes' emission process that converts low energy photons into high energy ones. During the past several decades, UC has attracted considerable attention accompanied with many proposed energy transfer mechanisms, such as excited state absorption (ESA), energy transfer upconversion (ETU), photon avalanche (PA), cooperative luminescence, etc. Among many Ln³⁺ ions, Er³⁺, Tm³⁺, Pr³⁺, Ho³⁺, and Nd³⁺ are excellent UC activators because of their abundant ladder-like energy levels with long-lived excited states, and Yb³⁺ is used as a common sensitizer in ETU systems due to its unique and simple energy level with only one excited level of ²F₅/2. However, UC efficiency is far from satisfactory due to the parity forbidden 4f-4f transitions and low absorption cross-sections of Ln³⁺ ions in addition to non-radiative processes induced by quenching of high frequency vibrations of surface ligands, such as hydroxyl groups, especially for nanoparticles (NPs). So far, the highest UC efficiency reported for NIR-to-visible conversion is only 4% and 5.1% for NaYF₄:Er³⁺,Yb³⁺ and NaYF₄:Er³⁺ under laser excitation of 20 and 0.27 W/cm², respectively. For visible-to-UVC conversion, it is only 0.001% from Y₂SiO₅:Pr³⁺,Gd³⁺,Li⁺ under 0.002 W/cm² excitation. Poor UC efficacy is attributed mainly to low absorption coefficients rather than inadequate quantum yields.

Therefore, many efforts have been made to enhance Ln³⁺ UC luminescence and eliminate non-radiative quenching. These include: introduction of non-Ln³⁺ ions to tailor local crystal field, plasmonic coupling with metal nanostructures, organic dyes as sensitizers. Although all these previous approaches can enhance the UC efficiency, many drawbacks still exist. Except the sensitization by dye molecules, all other approaches do not solve the fundamental issue of lanthanide UC, i.e. the inherently weak and narrowband absorption of Ln³⁺ ions. However, when they are employed as antenna ligands, the low photo- and thermal-stability and potential high toxicity of organic dye molecules are big concerns that pullback their practical applications. When noble metals are used to improve UC efficiency, their high material cost will prevent their wide deployment. Moreover, other than the only reported visible-to-UVC UC from Y₂SiO₅:Pr³⁺,Gd³⁺,Li⁺ with poor UC efficacy (0.001% under 0.002 W/cm² excitation), no other studies have been reported on adjusting absorption position of sensitizing materials to shorter wavelengths in the visible range.

Correspondingly, UC efficiency enhancement often occurs under NIR excitation to visible light emission. It is well known that the visible light is the most competent in sunlight. If the absorption of sensitizing antenna materials can be adjusted to visible region, they can dramatically enhance UC efficiency of lanthanide upconverters once excited by visible light, and solar energy is expected to be unitized efficiently as well. On the other hand, UV light has potential applications in UV solid-state laser, photocatalysis, and disinfection, etc. In sunlight, the UV light is only small fraction. If the visible and infrared light in sunlight can be efficiently converted into UV light, it will be very useful for these applications, especially photocatalysis and disinfection. Unfortunately, the UC efficiency into UV light is much lower because of narrow absorption section and requiring more photons, especially of NIR. Therefore, the UC issues of low absorptivity and narrow absorption spectrum of lanthanide upconverters, especially into the UV range, have not really been solved.

### SUMMARY OF THE INVENTION

To overcome the inherently weak and narrowband absorption of lanthanide ions due to the parity-forbidden nature of the 4f transitions, a new type of heterogeneous core@shell particles composed of a sensitizing persistent phosphor core and lanthanide upconversion (UC) shell has been developed to obtain high efficient upconversion materials. The enhancement of absorptivity and overall absorption spectrum and, therefore, UC efficiency of lanthanide UC materials is expected to have a significant positive impact in the areas of photocatalytic disinfection, solar cells, analyses and imaging techniques in medicine and diagnostics.

In an embodiment, an upconverting particle includes: a persistent phosphor core; and a lanthanide upconversion shell at least partially surrounding the persistent phosphor core. The emission wavelength of the persistent phosphor core is sufficient to create upconversion luminescence from the lanthanide upconversion shell.

In one embodiment, the persistent phosphor core has an emission wavelength sufficient to create upconversion luminescence from the lanthanide upconversion shell when exposed to visible light. In some embodiments, the persistent phosphor core has an emission wavelength greater than 650 nm. An exemplary persistent phosphor core is composed of ZnGa₂O₄:Cr³⁺. An exemplary lanthanide upconversion shell comprises NaYF₄:Ln³⁺ where Ln³⁺ is Tm³⁺.

The lanthanide upconversion shell produces light having a wavelength of less than 400 nm (e.g., ultraviolet light) in response to light emitted by the phosphor core. Thus, the particle produces light having a wavelength of less than 400 nm (e.g., ultraviolet light) in response to irradiation of the particle with light having a wavelength of between 400 nm and 700 nm (e.g., visible light).

A method of preparing an upconverting particle includes: obtaining a persistent phosphor core particle; and creating a coating at least partially surrounding the core particle, wherein the coating comprises a lanthanide upconversion shell. The persistent phosphor particle may be synthesized using a molten salt synthesis, hydrothermal synthesis, biphasic synthesis, or colloidal hot-injection synthesis. The persistent phosphor particle may be coated using a chimie douce (e.g. solvothermal) or a vapor phase coating process.

The upconverting particles described herein may be used in a photocatalytic water disinfection system. The photocatalytic water disinfection system may include a support; a plurality of upconverting particles as described in any one of claims 1-9 coupled to the support; and a light source capable of delivering activating light to the upconverting particles.

The upconverting particles described herein may be used in a solar energy cell. The solar energy cell may include a semiconductor substrate; a reflector coupled to the semiconductor substrate; and a plurality of upconverting particles positioned between the reflector and the semiconductor substrate.

The upconverting particles described herein may be used in a method of medical imagining and diagnostics. The method of medical imaging and diagnostics includes administering a plurality of upconverting particles to a subject; and irradiating the upconverting particles with activating light.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a schematic diagram of energy transfer in a core@shell particle composed of sensitizing persistent phosphor core and upconversion shell;

FIG. 2 depicts a schematic diagram of the synthesis of a core@shell particle composed of sensitizing persistent phosphor core and upconversion shell;

FIG. 3A depicts XRD results of the ZnGa₂O₄:Cr³⁺ core particles and upconverting core@shell particles having a ZnGa₂O₄:Cr³⁺ core and a NaYF₄:Tm³⁺ upconverting shell;

FIG. 3B depicts an SEM of a plurality of the upconverting core@shell particles having a ZnGa₂O₄:Cr³⁺ core and a NaYF₄:Tm³⁺ upconverting shell;

FIGS. 4A and 4B depict the change in broad band emission by varying the concentration of urea during the synthesis of the ZnGa₂O₄:Cr³⁺ core particles;

FIGS. 5A and 5B depict the change in decay time by varying the concentration of urea during the synthesis of the ZnGa₂O₄:Cr³⁺ core particles;

FIG. 6A shows the effect of chromium concentration on the emission of the ZnGa₂O₄:Cr³⁺ core particles having a Zn to Ga ratio of 1:1;

FIG. 6B shows the effect of chromium concentration on the decay time of the ZnGa₂O₄:Cr³⁺ core particles having a Zn to Ga ratio of 1:1;

FIG. 7A shows the effect of hexamethylenetetramine (HMTA) concentration on the emission of the ZnGa₂O₄:Cr³⁺ core particles having a Zn to Ga ratio of 1:1;

FIG. 7B shows the effect of HMTA concentration on the decay time of the ZnGa₂O₄:Cr³⁺ core particles having a Zn to Ga ratio of 1:1;

FIG. 8A shows the effect of ammonium nitrate concentration on the emission of the ZnGa₂O₄:Cr³⁺ core particles having a Zn to Ga ratio of 1:1;

FIG. 8B shows the effect of ammonium nitrate concentration on the decay time of the ZnGa₂O₄:Cr³⁺ core particles having a Zn to Ga ratio of 1:1;

FIG. 9A shows an XRD analysis of the ZnGa₂O₄:Cr³⁺ core particles having a Zn to Ga ratio of 1:2 and urea concentrations varying from 5 mmol to 100 mmol;

FIG. 9b shows an XRD analysis of the ZnGa₂O₄:Cr³⁺ core particles having a Zn to Ga ratio of 1:1 and urea concentrations varying from 5 mmol to 50 mmol;

FIG. 10A shows that the formation of a zinc oxide shell can enhance the emission of the ZnGa₂O₄:Cr³⁺ core particles;

FIG. 10B shows that the formation of a zinc oxide shell can enhance the decay time of the ZnGa₂O₄:Cr³⁺ core particles;

FIG. 11A shows the emission spectrum of the ZnGa₂O₄:Cr³⁺ core particles under 405 nm excitation and the absorption spectrum of NaYF₄:Tm³⁺;

FIG. 11B is the UC spectra of the ZnGa₂O₄:Cr³⁺ core and the ZnGa₂O₄:Cr³⁺@NaYF₄:Tm³⁺ core@shell nanoparticles; and

FIG. 11C depicts a schematic diagram of energy transfer during an upconversion process from the ZnGa₂O₄:Cr³⁺@NaYF₄:Tm³⁺ core@shell particles composed of sensitizing persistent phosphor core and upconversion shell.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

As used herein “nanoparticle” refers to a particle having a size of less than 1 micron.

As used herein the term “upconversion (UC)” refers to a process in which the adsorption of photons leads to the emission of light at a shorter wavelength than the excitation wavelength.

As used herein “activating light” means light that may cause an upconversion effect in an upconverting particle.

As used herein a “persistent phosphor” is a phosphorescent material that has persistent emission of light for at least 1 second after irradiation with activating light, either broadband or narrowband.

Described herein are heterogeneous core@shell particles composed of a sensitizing persistent phosphor core and a UC shell. The core@shell particles can be used for upconversion of visible light to ultraviolet light.

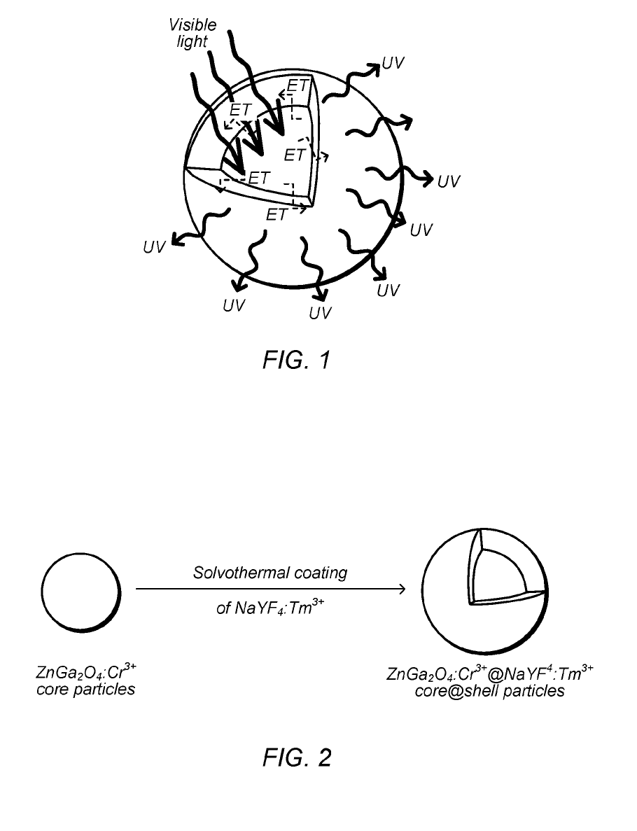

Long-persistent phosphor-sensitized core@shell UC materials are described herein. As previously discussed, for UV UC emissions, the efficiency is very low even for widely used upconverting Ln³⁺ ions including Tm³⁺, Ho³⁺, Er³⁺, and Pr³⁺ because these ions require absorbing more than four NIR photons before excitation occurs. Additionally, these upconverters also have inherently weak and narrow band absorption. In the embodiments described herein the UV UC efficiency can be greatly improved via energy transfer from the core to the shell by core@shell particles. A schematic diagram of energy transfer in a core@shell particle composed of sensitizing persistent phosphor core and upconversion shell is shown in FIG. 1. The core has broad absorption in the visible region and strong emission with spectral overlap over the excitation of Ln³⁺ ions in the shell. The spectral overlap and energy match assist in achieving efficient energy transfer.

Persistent phosphors are used as the core material of the core@shell particle. Generally, persistent phosphors are composed of a host material and an activator disposed in the host material. Exemplary materials that can be used to form a persistent phosphor particle include but are not limited to: CaMgSi₂O₆:Mn²⁺; BaMg₂Si₂O₇:Mn²⁺; Ca₂MgSi₂O₇:Dy³⁺; Sr₂MgSi₂O₇:Dy³⁺; Ca₃MgSi₂O₈:Dy³⁺; Ca₃SnSi₂O₉:Dy³⁺; SrSiO₃:Dy³⁺; Sr₂SiO₄:Dy³⁺; CaAl₂O₄:Dy³⁺; MgAl₂O₄:Cr³⁺; Ca₂SnO₄:Sm³⁺; Sr₂SnO₄:Sm³⁺; CaSnO₃:Sm³⁺; Sr₃Sn₂O₇:Mn²⁺; Ca₉Gd(PO₄)₇:Mn²⁺; Ca₉Lu(PO₄)₇:Mn²⁺; Ca₃(PO₄)₂:Mn²⁺; Gd₃Ga₅O₁₂:Cr³⁺; LiGa₅O₈:Cr³⁺; ZnGa₂O₄:Cr³⁺; MgGeO₃:Mn²⁺; La₃Ga₅GeO₁₄:Cr³⁺; Zn₃Ga₂Ge₂O₁₀:Cr³⁺; Y₂O₂S:Eu³⁺; and Y₂O₂S:Sm³⁺.

The persistent phosphor is selected such that the emission spectrum of the persistent phosphor matches the activation wavelength(s) of the upconversion shell. For example, UC luminescence from Ln³⁺ ions in the shell can usually be excited under long wavelength (>650 nm). So the emission wavelength of the core material would, in these instances, be longer than 650 nm.

An exemplary persistent phosphor is ZnGa₂O₄:Cr³⁺ which can sensitize UC luminescence of Ln³⁺ ions because of their strong broad band emission (>650 nm) under visible light excitation. In addition, the Cr³⁺ emission can be adjusted by adding Ge⁴⁺ to the host material giving a host material having the structure Zn(Ga₁₋ₓGeₓ)₂O₄ where Ge⁴⁺ ions replace some of the Ga⁴⁺ ions. More than being able to overcome the issues inherited with previous approaches as discussed above, these inorganic persistent phosphors with high stability and low/no toxicity are expected to act as ideal sensitizers to increase absorptivity and broaden absorption spectrum of Ln³⁺ upconverters. The advantages of combining long-persistent phosphor antennas with Ln³⁺ upconverters include (i) the strong visible light absorption, (ii) the broad absorption spectrum, (iii) the ability to tune the absorption band by bandgap engineering, (iv) the possibility of using a set of complementary antenna phosphors to further optimize the absorption range, (v) the photo- and thermal-stability in addition to low/non-cytotoxicity of both the inorganic antenna phosphors and upconverters, and (vi) still active after turning off excitation.

Lanthanide upconverter materials include a variety of host materials doped with one or more lanthanides. The phonon energy of the hosts for Ln³⁺ ions can also influences UC luminescent efficiency. Exemplary lanthanide upconverters include, but are not limited to: NaYF₄:Ln³⁺; GdVO₄:Ln³⁺; Lu₂O₃:Ln³⁺; Gd₄O₃F₆:Ln³⁺; NaYbF₄:Ln³⁺; CaF₂:Ln³⁺; Gd₄O₃F₆:Ln³⁺; BaTiO₃:Ln³⁺; La₂O₃:Ln³⁺; Y₂O₃:Ln³⁺; and Lu₂O₃:Ln³⁺. Ln³⁺ represents one or more lanthanide dopant atoms dispersed in the host material. The term “lanthanide” refers to the elements having an atomic number from 57 through 71. Exemplary lanthanides that are particularly useful for use in an upconverting layer include, but are not limited to: Tm³⁺; Ho³⁺; Er³⁺; and Pr³⁺. To eliminate non-radiative quenching processes from the surface exposed Ln³⁺ ions, to enhance the overall upconverting efficiency, part of the shell, more specifically, the outmost shell contains no Ln³⁺ ions. This can be done by altering the lanthanide doping of the shell during synthesis.

The described core@shell particles overcome the inherently weak and narrowband absorption of Ln³⁺ ions due to the parity-forbidden nature of the 4f transitions. As well-known, these limitations have seriously hampered the utility of UC materials for practical applications. By fundamentally developing a novel “sensitizing” strategy by long-persistent phosphors to enable an increase in absorptivity and overall absorption spectrum of lanthanide upconverters, the embodiments described herein contribute to a markedly improved UC material system that integrates increased absorptivity and broad overall absorption spectrum. This contribution is significant, because it is expected to change UC from an academic and exotic phenomenon into a realistic and viable tool for increasing the efficiencies of many devices. For example, the enhancement of absorptivity and overall absorption spectrum and therefore UC efficiency of lanthanide UC materials is expected to have significant positive impact in the areas of photocatalytic disinfection, solar cells, analyses and imaging techniques in medicine and diagnostics. In other words, it is expected that it will become possible to widely deploy UC materials in drinking water disinfection, solar energy conversion, consumer optoelectronics, medical imaging and diagnostics.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

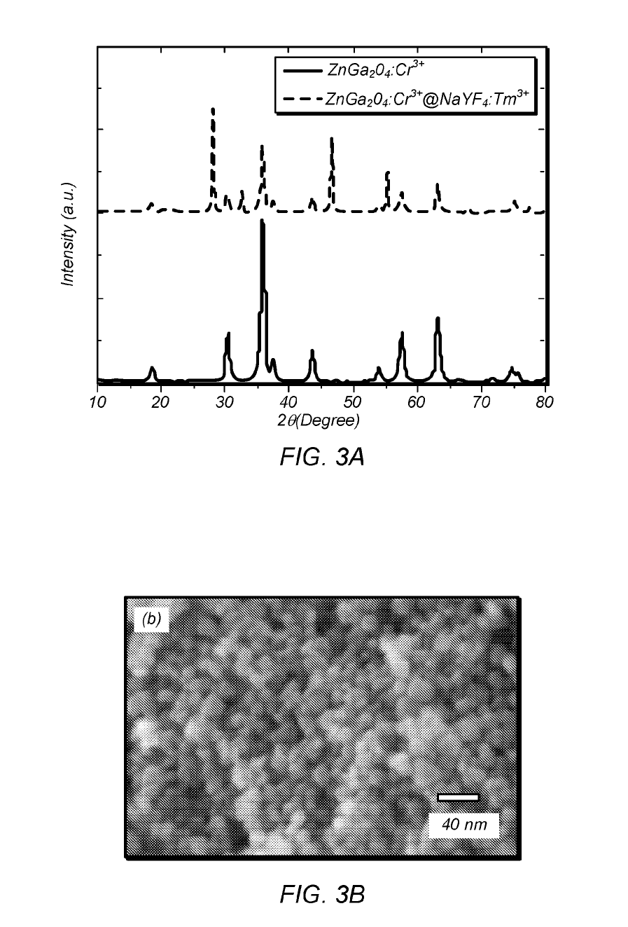

NaYF₄ is the most efficient host materials for UC. So Ln³⁺ doped NaYF₄ will be used as the shell in this project as an example. To investigate long-persistent phosphor-sensitized core@shell UC particles, ZnGa₂O₄:Cr³⁺ particles were first synthesized by molten salt synthesis, hydrothermal synthesis, biphasic synthesis, or colloidal hot-injection synthesis, and then were coated with a thin layer of NaYF₄:Tm³⁺ by a chimie douce (e.g. solvothermal) or a vapor phase method to form the ZnGa₂O₄:Cr³⁺@NaYF₄:Tm³⁺ core@shell particles. A schematic diagram of this reaction is shown in FIG. 2. XRD results (shown in FIG. 3A) confirmed the formation of ZnGa₂O₄:Cr³⁺ and NaYF₄:Tm³⁺ composite after coating the ZnGa₂O₄:Cr³⁺ core with NaYF₄:Tm³⁺. The average ZnGa₂O₄:Cr³⁺ core particle size is around 5-100 nm and the shell thickness can be controlled during the coating process (see FIG. 3B).

Synthesis of ZnGa₂O₄:Cr³⁺ Particles

A general sequence for the hydrothermal synthesis of ZnGa₂O₄:Cr³⁺ particles is shown below:

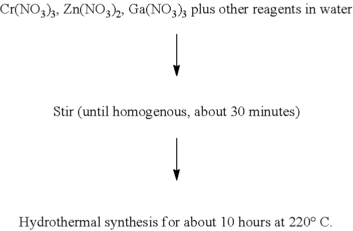

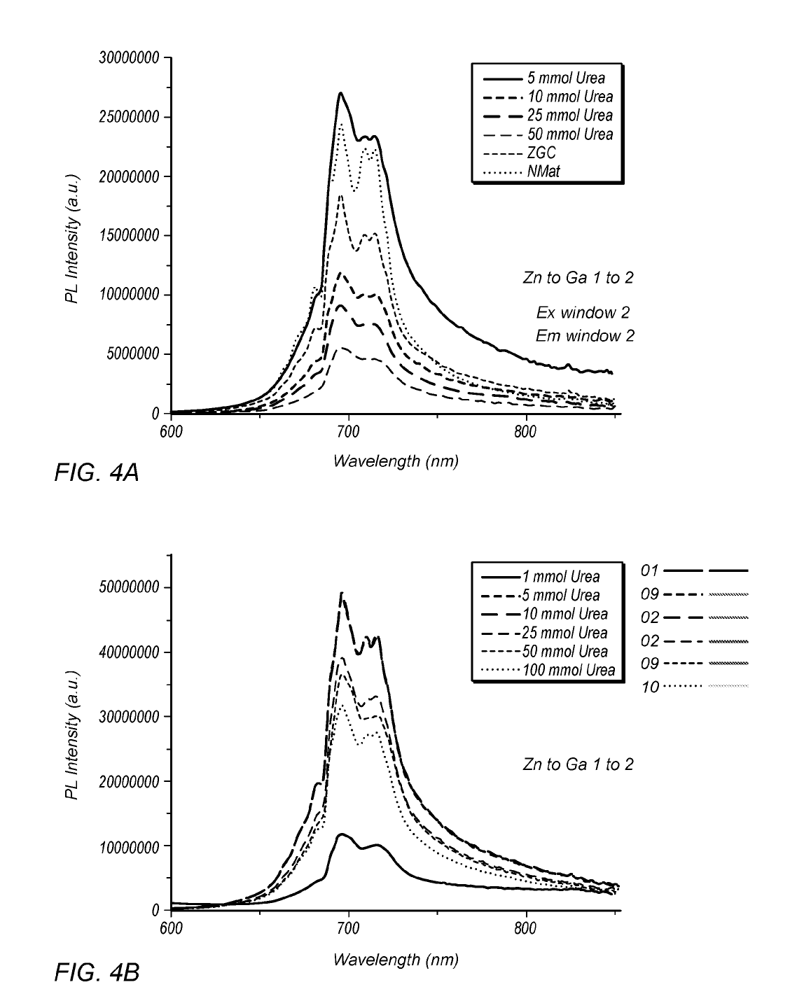

“Other reagents” include one or more of the following: urea, ammonium nitrate, and hexamethylenetetramine (HMTA). Various reaction parameters were investigated to determine the optimal process for preparing the ZnGa₂O₄:Cr³⁺ particles. Some of the reaction parameters investigated include: ammonium nitrate concentration; ratio of the metal nitrates of Zn and Ga; chromium doping concentration (relative amount of Cr to Zn and Ga); urea concentration, and HMTA concentration (FIGS. 4-10).

The persistent phosphor ZnGa₂O₄:Cr³⁺ core particles can sensitize UC luminescence of Ln³⁺ ions because of its strong broad band emission (>650 nm) under visible light excitation. The effect of various process parameters on the emission strength and the decay time was studied.

FIGS. 4A and 4B depict the change in broad band emission and decay time by varying the concentration of urea during the synthesis of the ZnGa₂O₄:Cr³⁺ core particles. In FIG. 4A the ratio of Zn to Ga was set at 1:2, while the concentration of urea was varied from 5 mmol up to 50 mmol. ZGC refers to the ZnGa₂O₄:Cr³⁺ core particles made in the absence of urea. “NMat” refers to the compound described in Maldiney et al, “The in vivo activation of persistent nanophosphors for optical imaging of vascularization, tumors and grafted cells” Nature Materials 2014, 13, 418-426. “JACS” refers to a compound described in Li et al. “Direct Aqueous-Phase Synthesis of Sub-10 nm ‘Luminous Pearls’ with Enhanced in Vivo Renewable Near-Infrared Persistent Luminescence” Journal of the American Chemical Society, 2015, 137 (16), pp 5304-5307. As can be seen in FIG. 4A, setting the initial concentration at 5 mmol 10 mmol of urea during synthesis of the ZnGa₂O₄:Cr³⁺ core particles provides particles having the greatest broad band emission. In FIG. 4B the ratio of Zn to Ga was set at 1:1, while the concentration of urea was varied from 5 mmol up to 100 mmol. As was seen in FIG. 4A, FIG. 4B shows that the optimal emission is obtained at concentrations of urea of 5 mmol 10 mmol.

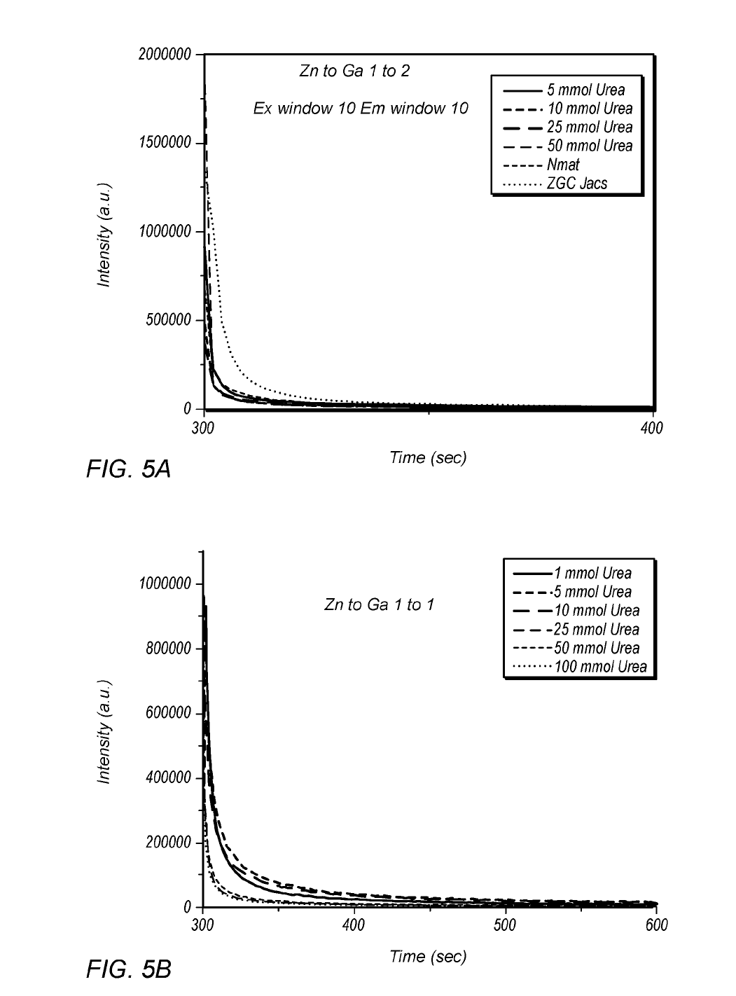

FIGS. 5A and 5B depict the change in decay time by varying the concentration of urea during the synthesis of the ZnGa₂O₄:Cr³⁺ core particles. When the ratio of Zn to Ga is set at 1:2, varying the concentration of urea has little effect on the decay time, as shown in FIG. 5A. In FIG. 5B, where the concentration of Zn to Ga was set at 1:1, the concentration of urea has a significant effect on the decay time. Specifically, concentrations of 5 mmol to 10 mmol of urea lead to the longest decay times of the ZnGa₂O₄:Cr³⁺ core particles.

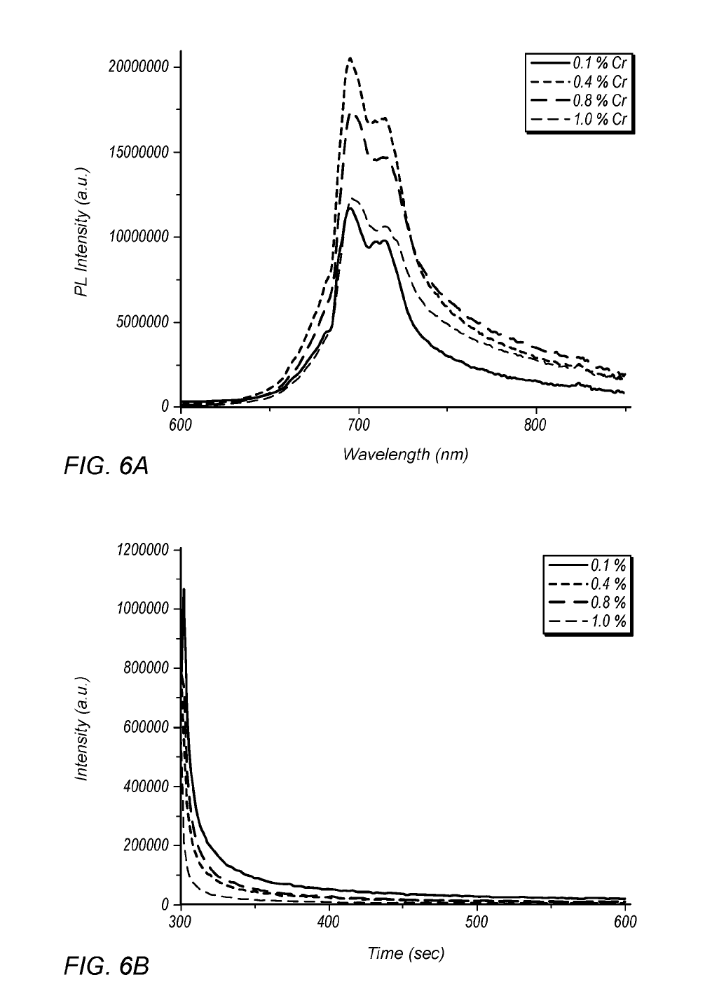

FIGS. 6A and 6B show the effect of chromium concentration on the emission (FIG. 6A) and the decay time (FIG. 6B) of the ZnGa₂O₄:Cr³⁺ core particles having a Zn to Ga ratio of 1:1. FIG. 6A shows that an optimal concentration, with respect to emission, is seen at a Cr concentration (in the initial mixture) of between 0.4% Cr to about 0.8% Cr. FIG. 6B depicts the effect of chromium concentration on the decay time of the ZnGa₂O₄:Cr³⁺ core particles.

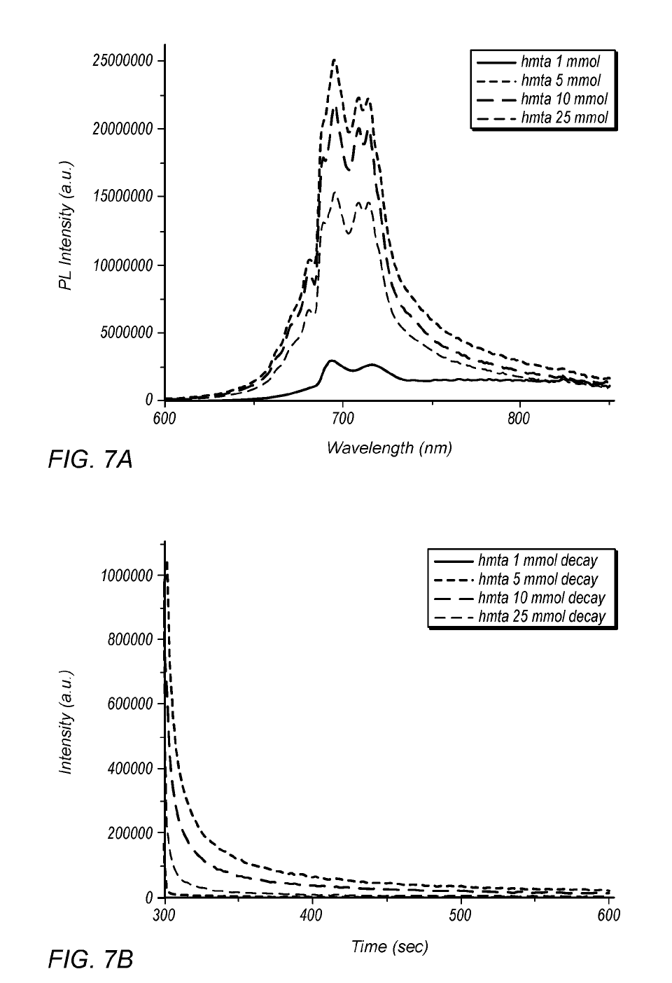

FIGS. 7A and 7B show the effect of HMTA concentration on the emission (FIG. 7A) and the decay time (FIG. 7B) of the ZnGa₂O₄:Cr³⁺ core particles having a Zn to Ga ratio of 1:1. FIG. 7A shows that an optimal concentration, with respect to emission, is seen at a HMTA concentration (in the initial mixture) of between 5 mmol to 10 mmol. FIG. 7B depicts the effect of HMTA concentration on the decay time of the ZnGa₂O₄:Cr³⁺ core particles.

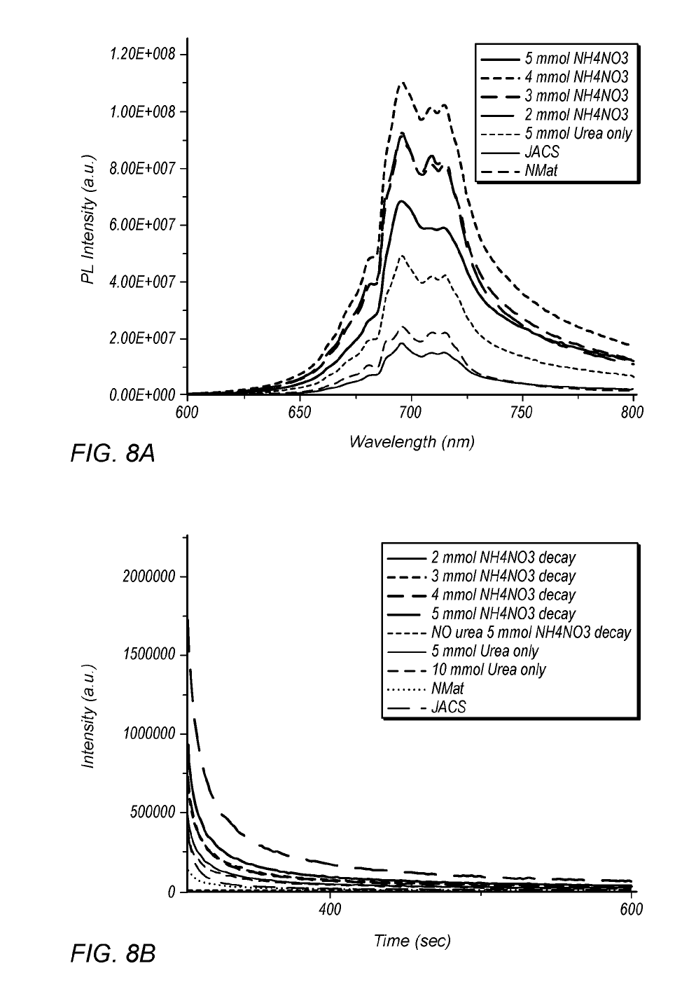

FIGS. 8A and 8B show the effect of ammonium nitrate concentration on the emission (FIG. 8A) and the decay time (FIG. 8B) of the ZnGa₂O₄:Cr³⁺ core particles having a Zn to Ga ratio of 1:1. FIG. 8A shows that an optimal concentration, with respect to emission, is seen at an ammonium nitrate concentration (in the initial mixture) of between 2 mmol to 4 mmol. FIG. 8B depicts the effect of ammonium nitrate concentration on the decay time of the ZnGa₂O₄:Cr³⁺ core particles.

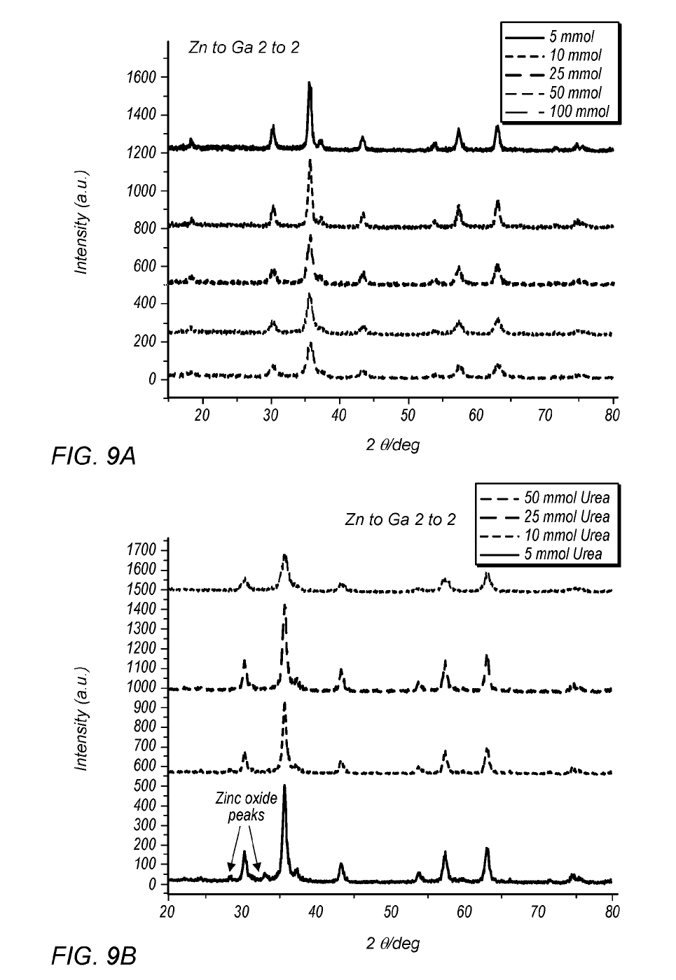

During TEM imaging, it was noted that some ZnGa₂O₄:Cr³⁺ core particle samples prepared from precursor solution containing urea, when the ratio of Zn to Ga was 1:1, developed a core-shell formation, possibly due to the excess zinc present in the initial solution forming a zinc oxide coating on the ZnGa₂O₄:Cr³⁺ core particles. This was verified by using XRD analysis to show the presence of zinc oxide when the ratio of Zn to Ga was increased from 1:2 to 1:1. The XRD analysis is shown in FIG. 9A (Zn to Ga ratio 1:2, urea concentrations varying from 5 mmol to 100 mmol) and FIG. 9B (Zn to Ga ratio 1:1, urea concentrations varying from 5 to 50 mmol urea. FIG. 9B shows the development of ZnO peaks when the urea concentration is about 5 mmol.

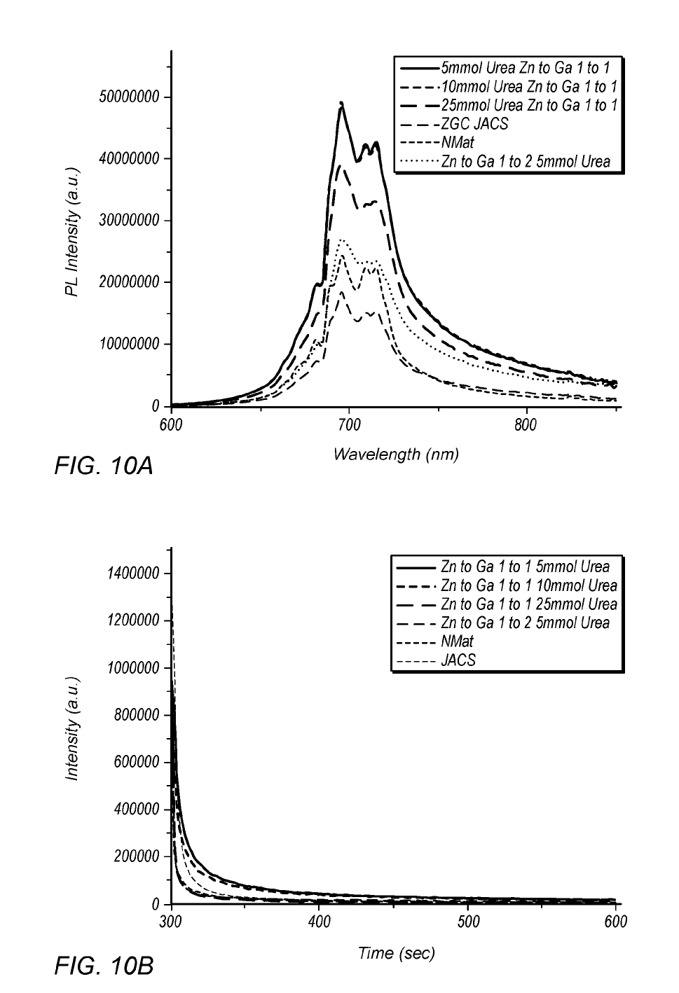

FIGS. 10A and 10B show that the formation of a zinc oxide shell can enhance the emission and decay time of the ZnGa₂O₄:Cr³⁺ core particles. FIG. 10A shows that emission is greatest in particles formed under conditions that promote zinc oxide shell formation (excess zinc (Zn to Ga 1:1) and low urea concentrations (5 mmol to 10 mmol). FIG. 10B shows that the decay time is also enhanced by the formation of a zinc oxide shell.

It is believed that a shell of zinc oxide improves the emission and increased life-time of persistent luminescence due to a lattice defect and decrease in surface defects. In addition, a correlation between particles' size is seen to increase particles' emission and decay time due to the replacement of the doping material and the amount of surface defects.

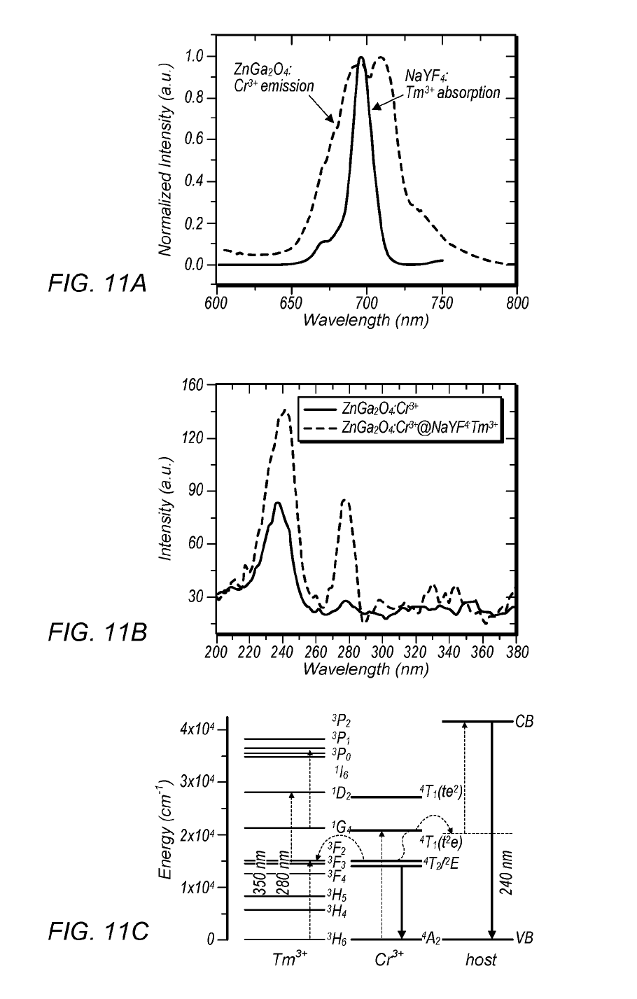

FIG. 11A shows the emission spectrum of the ZnGa₂O₄:Cr³⁺ core particles under 405 nm excitation and the absorption spectrum of NaYF₄:Tm³⁺. An obvious spectral overlap is observed, which is expected to allow efficient energy transfer from the ZnGa₂O₄:Cr³⁺ core to the NaYF₄:Tm³⁺ shell. FIG. 11B is the corresponding UC spectra of the ZnGa₂O₄:Cr³⁺ core and ZnGa₂O₄:Cr³⁺@NaYF₄:Tm³⁺ core@shell nanoparticles. When ZnGa₂O₄:Cr³⁺@NaYF₄:Tm³⁺ core@shell nanoparticles were excited by 405 nm laser, two strong peaks at 240 and 280 nm as well as a weak peak at 350 nm were observed. The emissions at 280 and 350 nm are attributed to the ³P₀→³H₆ and ¹D₂→³H₆ transitions of Tm³⁺ ions, respectively. The occurrence of these two emission peaks confirms the existence of energy transfer from the ZnGa₂O₄:Cr³⁺ core to the NaYF₄:Tm³⁺ shell since the latter cannot be directly excited by 405 nm laser. The emission at 240 nm corresponds to the emission of the ZnGa₂O₄ host. The detailed UC processes are shown in FIG. 11C. Cr³⁺ ions are excited by 405 nm laser from the ⁴A₂ ground state to the ⁴T₁ (t²e) excited state. Then it will relax to the ⁴T₂/²E state via non-radiation process. Because of the large spectral overlap between the emission of the ⁴T₂/²E state of Cr³⁺ ions and the absorption of the ³F_{3,2} levels of Tm³⁺, efficient energy transfer from the ⁴T₂/²E state of Cr³⁺ ions to the ³F_{3,2} level of Tm³⁺ ions occurs, which populates the ³F_{3,2} level of Tm³⁺ ions. After absorbing another photon from the ⁴T₂/²E state of Cr³⁺ ions, the excited Tm³⁺ ions can be further excited from the ³F₃ level to ¹D₂ level, following a 350 nm emission from the ¹D₂ level to the ground state. In another process, the population can also relax from ¹D₂ level to ¹G₄ level, resulting in excitable ¹G₄ level. After absorbing a photon from ⁴T₂/²E state of Cr³⁺ ions, the Tm³⁺ ions on the ¹G₄ level can be further excited to the ³P₀ level, resulting in a 280 nm emission.

Regarding the emission at 240 nm, cooperative luminescent mechanism should be responsible. That means, after absorbing two photons from ⁴T₁ (t²e) state, electrons can be excited from valance band (VB) of the semiconductor ZnGa₂O₄ host to its conduction band (CB). The return of excited electrons from the CB of ZnGa₂O₄ to its VB gives the emission at 240 nm. These results confirm the feasibility of Ln³⁺ UC can be sensitized by persistent phosphors.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. (canceled)

2. An upconverting particle comprising:
an inorganic long-persistent phosphor core; and
a lanthanide upconversion shell at least partially surrounding the persistent phosphor core;
wherein an emission wavelength of the persistent phosphor core is sufficient to create upconversion luminescence from the lanthanide upconversion shell.

3. The upconverting particle of claim 2, wherein the persistent phosphor core has an emission wavelength sufficient to create upconversion luminescence from the lanthanide upconversion shell when exposed to either broadband light such as visible light or narrowband light such as laser.

4. The upconverting particle of claim 2, wherein the persistent phosphor core has an emission wavelength greater than 400 nm.

5. The upconverting particle of claim 2, wherein the persistent phosphor core comprises ZnGa₂O₄:Cr³⁺/Mn²⁺ or Zn(Ga₁₋ₓGeₓ)₂O₄:Cr³⁺/Mn²⁺.

6. The upconverting particle of claim 2, wherein the lanthanide upconversion shell comprises NaYF₄:Ln³⁺, YBO₃:Ln³⁺, or Y₂O₃:Ln³⁺.

7. The upconverting particle of claim 2, wherein the persistent phosphor core comprises ZnGa₂O₄:Cr³⁺/Mn²⁺ or Zn(Ga₁₋ₓGeₓ)₂O₄:Cr³⁺/Mn²⁺, and wherein the lanthanide upconversion shell comprises NaYF₄:Ln³⁺, YBO₃:Ln³⁺, or Y₂O₃:Ln³⁺.

8. The upconverting particle of claim 2, wherein an outmost portion of the lanthanide upconversion shell contains substantially no Ln³⁺ ions.

9. The upconverting particle of claim 7, wherein Ln³⁺ is a “lanthanide” having an atomic number from 57 through 71 or their mixture.

10. The upconverting particle of claim 2, wherein the lanthanide upconversion shell produces light having a wavelength of less than 650 nm in response to light emitted by the persistent phosphor core.

11. The upconverting particle of claim 2, wherein the particle produces light having a wavelength of shorter than 650 nm in response to irradiation of the core@shell particle to light having a wavelength longer than 400 nm.

12. A method of preparing an upconverting core@shell particle comprising:
obtaining an inorganic long-persistent phosphor core particle;
creating a coating at least partially surrounding the core particle, wherein the coating comprises a lanthanide upconversion shell;
wherein an emission wavelength of the persistent phosphor core is sufficient to create upconversion luminescence from the lanthanide upconversion shell.

13. The method of claim 12, wherein obtaining a persistent phosphor particle comprises using a molten salt synthesis, hydrothermal synthesis, biphasic synthesis, or colloidal hot-injection synthesis to prepare the persistent phosphor particle.

14. The method of claim 12, wherein creating a coating at least partially surrounding the core particle comprises using a chimie douce (e.g. solvothermal) or a vapor phase coating process to form a core@shell particle.

15. The method of claim 12, wherein the persistent phosphor particle comprises ZnGa₂O₄:Cr³⁺/Mn²⁺ or Zn(Ga₁₋ₓGeₓ)₂O₄:Cr³⁺/Mn²⁺.

16. The method of claim 12, wherein the lanthanide upconversion shell comprises NaYF₄:Ln³⁺, YBO₃:Ln³⁺, or Y₂O₃:Ln³⁺.

17. The method of claim 16, wherein Ln³⁺ is a “lanthanide” having an atomic number from 57 through 71 or their mixture.

18-20. (canceled)
